(19) United States
(12) Patent Application Publication
(10) Pub. No.: US 2006/0161121 A1
(43) Pub. Date: Jul. 20, 2006

(54) Peroxide compositions

(75) Inventors: Jo Klaveness, Krakeroy (NO); Thomas Cleve, Fragner (NO); Kenneth Finstad, Krakeroy (NO); Bjame Brudeli, Krakeroy (NO)

Correspondence Address: BACON & THOMAS, PLLC, 625 SLATERS LANE, FOURTH FLOOR, ALEXANDRIA, VA, 22314, US

(73) Assignee: DEHLI-CLEVE COLLECTION AS, KRAKEROY (NO)

(21) Appl. No.: 10/544,362

(22) PCT Filed: Feb. 6, 2004
(86) PCT No.: PCT/GB04/00481

§ 371 Date: Mar. 14, 2006

(30) Foreign Application Priority Data
Feb. 6, 2003 (GB) 0302739.8
Sep. 5, 2003 (GB) 0320863.4

(51) Int. Cl. A61M 35/00

(52) U.S. Cl. 604/289

(57) ABSTRACT

A kit comprising: a sealed container containing a ready-to-use skin bleaching composition including an emulsion containing 2 to 6% wt of a peroxide bleaching agent selected from hydrogen peroxide, organic peracids and salts thereof, hydrogen peroxide adducts, and metal peroxides, percarbonates and perborates; and instructions for the use of the composition to bleach chemically tanned skin.

This invention relates to peroxide containing compositions useful for bleaching chemically induced tan from the skin.

Tanning, i.e. browning, of the skin can be induced by application of chemical tanning agents such as for example dihydroxyacetone (DHA) (see Levy in J. Am. Acad. Dermatol 29:284-286 (1993) and U.S. Pat. No. 6,231,837). With time, the artificial tan will naturally fade. However some users of artificial tanning agents, for example participants in dancing or body-building competitions, wish to remove the artificial tan without waiting for it to fade.

In U.S. Pat. No. 6,117,118, U.S. Pat. No. 6,322,544 and U.S. Pat. No. 6,235,783 Laughlin Products Inc disclosed a process for bleaching chemically tanned skin that involved application either of a simple 3% solution of hydrogen peroxide or alternatively of a two-component hair bleaching cream comprising a hydrogen peroxide-containing cream and a separate ammonium bicarbonate or hydroxide containing activator.

We have investigated the utility of the simple 3% aqueous solution of hydrogen peroxide and have found that it is virtually ineffective at bleaching chemically tanned skin. Indeed Laughlin et al admit this in their patents and instead present the two-component systems as being the effective ones.

However, especially where a large area of skin has to be treated, a two-component composition is highly inconvenient to the user.

We have now surprisingly found that a single component ready-to-use peroxide containing composition emulsion may be used to efficiently remove artificial tan.

We have now also surprisingly found that bleaching of chemically tanned skin may be improved if rather than using hydrogen peroxide as the or the only peroxide bleaching agent, other peroxides are instead or additionally used. Typically such other peroxides may be selected from hydrogen peroxide urea adducts, metal peroxides and metal percarbonates, in particular group I or II peroxides and percarbonates.

Moreover, we have also found that bleaching of chemically tanned skin may suitably be effected using a single component (i.e. ready-to-use) system which comprises a composition which is physicochemically compartmented having a continuous compartment and a discontinuous compartment (e.g. an emulsion or a dispersion of fragmented liquid crystalline structures or readily frangible coated particles) with one compartment containing a peroxide (e.g. hydrogen peroxide or such other peroxides as are discussed herein) and a different compartment containing a peroxide activator (e.g. an alkali or a peroxide compound or di or multivalent metal ions or a source thereof).

On application of such a ready-to-use single-component system to the skin and rubbing it in, compartmentalization is broken down and the peroxide is activated causing the bleaching effect to be enhanced.

Viewed from one aspect therefore the invention provides a kit comprising: a sealed container containing a ready-to-use skin bleaching composition comprising an emulsion containing 2 to 6% wt of a peroxide bleaching agent selected from the group consisting of hydrogen peroxide, organic peracids and salts thereof, hydrogen peroxide adducts (in particular hydrogen peroxide-urea adducts, hydrogen peroxide-metal phosphate adducts (e.g. pyrophosphate or tripolyphosphate adducts)), and metal peroxides, percarbonates and perborates (in particular group I or II (i.e. alkali metal or alkaline earth metal) peroxides, percarbonates and perborates, especially sodium, potassium, calcium or magnesium compounds); and instructions for the use of said composition to bleach chemically tanned skin.

In the kit of the invention, the peroxide bleaching agent is preferably hydrogen peroxide or a hydrogen peroxide adduct, especially hydrogen peroxide.

It should be noted that the compositions described in U.S. Pat. No. 6,117,118, U.S. Pat. No. 6,322,544 and U.S. Pat. No. 6,236,783 are either not emulsions or are not ready-to-use in the form in which they are packaged.

Viewed from a further aspect the invention provides a method of cosmetic treatment to bleach chemically tanned skin, said method comprising obtaining a kit according to the invention and applying said composition to chemically tanned skin.

Viewed from a further aspect the invention provides a method of cosmetic treatment to bleach chemically tanned skin, said method comprising applying to chemically tanned skin a topically acceptable emulsion which has a neutral or acid pH and comprises 2 to 6% wt of a peroxide bleaching agent selected from the group consisting of hydrogen peroxide, organic peracids and salts thereof, hydrogen peroxide adducts (in particular hydrogen peroxide-urea adducts, hydrogen peroxide-metal phosphate adducts (e.g. pyrophosphate or tripolyphosphate adducts)), and metal peroxides, percarbonates and perborates (in particular group I or II (i.e. alkali metal or alkaline earth metal) peroxides, percarbonates and perborates, especially sodium, potassium, calcium or magnesium compounds).

Viewed from a still further aspect the invention provides a method of cosmetic treatment to bleach chemically tanned skin, said method comprising applying to chemically tanned skin a topically acceptable emulsion which comprises 2 to 6% wt of a peroxide bleaching agent selected from the group consisting of hydrogen peroxide, organic peracids and salts thereof, hydrogen peroxide adducts (in particular hydrogen peroxide-urea adducts, hydrogen peroxide-metal phosphate adducts (e.g. pyrophosphate or tripolyphosphate adducts)), and metal peroxides, percarbonates and perborates (in particular group I or II (i.e. alkali metal or alkaline earth metal) peroxides, percarbonates and perborates, especially sodium, potassium, calcium or magnesium compounds) and which emulsion is essentially free of alkali hydrogen peroxide activators, i.e. where present any such activator must be at a concentration insufficient to reduce the peroxide bleaching agent concentration below 2% wt on storage at 21° C. in the dark for six weeks.

The compositions of the invention are conveniently in a ready-to-use form, i.e. they can be applied directly to the skin without requiring further mixing following removal from the containers in which they are packaged.

These compositions of the invention are emulsions, optionally oil-in-water emulsions but preferably water-in-oil emulsions. The oil phase may be any topically tolerable oil or oil mixture but preferably comprises both hydrophobic and amphiphilic constituents. The use of liquid paraffin as at least one component of the oil phase is especially preferred.

The oil phase to aqueous phase ratio is preferably such that the emulsion composition has a creamy consistency rather than a paste or liquid like consistency.

The composition is preferably packed in a sealed pot, jar, bottle, tube or sachet, especially a squeezable plastic tube or bottle, for example of the type used for containing toothpaste or shampoo. Sealing of the container may for example be by way of a removable lid or a breakable foil. The container is preferably opaque.

Unlike the two component compositions of U.S. Pat. No. 6,117,118, U.S. Pat. No. 6,322,544 and U.S. Pat. No. 6,225,783 above, these compositions of the invention do not require admixture of an alkaline activator. Indeed, the compositions of the invention are preferably either neutral or acidic, e.g. pH 3 to 7, especially pH 4.5 to 6.5, particularly pH 5 to 6.

The use of peroxide bleaching agents as described above other than hydrogen peroxide itself is also new and forms a further aspect of the invention. Thus viewed from this further aspect the invention provides a composition for the bleaching of chemically tanned skin containing a peroxide bleaching agent and a physiologically tolerable carrier, characterized in that said peroxide bleaching agent is selected from the group consisting of organic peracids and salts thereof, hydrogen peroxide adducts (in particular hydrogen peroxide-urea adducts, hydrogen peroxide—metal phosphate adducts (e.g. pyrophosphate or tripolyphosphate adducts)), and metal peroxides, percarbonates and perborates (in particular group I or II (i.e. alkali metal or alkaline earth metal) peroxides, percarbonates and perborates, especially sodium, potassium, calcium or magnesium compounds).

In such compositions, where the peroxide bleaching agent is a metal salt, the metal is preferably sodium or calcium. The peroxide bleaching agent however especially preferably comprises carbamyl peroxide, i.e. a hydrogen peroxide—urea adduct. If desired the composition may also contain hydrogen peroxide. The total peroxide content of the composition is desirably 2 to 6% wt., especially 3 to 5% wt.

Such compositions are especially preferably formulated as ready-to-use emulsions (e.g. as described above) or as physicochemically compartmented forms as described herein.

Such compositions moreover are preferably presented in kit form comprising a container containing the composition and instructions for use in treatment of the skin to bleach chemically tanned skin. Such kits form a further aspect of the present invention.

Viewed from a yet further aspect the invention also provides a ready-to-use skin bleaching composition for bleaching chemically tanned skin, said composition being compartmentalized having a continuous compartment and a discontinuous compartment with one of said compartments containing a peroxide bleaching agent (e.g. hydrogen peroxide and/or one of the other peroxides mentioned above) and the other of said compartments containing a peroxide activator (e.g. an alkali or a further peroxide compound or di or multivalent cations or a source thereof, e.g. calcium peroxide).

Compartmentalization in such bleaching compositions may be achieved by conventional means, e.g. using fragmented liquid crystalline phases (e.g. liposomes, micelles, or cubic or hexagonal phase structures (see for example U.S. Pat. No. 5,531,925 and EP-A-867168)), emulsions, frangible microspheres, frangible coated particulates, etc.

In such compartmentalized compositions, the peroxide content is preferably 2 to 6% wt., especially 3 to 5% wt., and the activator concentration is preferably 0.05 to 10% wt., especially 0.1 to 5% wt.

The remaining components of the composition may be ones conventionally used in cosmetics and topical pharmaceuticals, e.g. as carriers, diluents, stabilizing agents, pH modifiers, buffers, aromas, viscosity modifiers, etc.

Preferably however the peroxide bleaching agent is present in a continuous aqueous phase in the compositions and the peroxide bleaching agent-containing compartment preferably is at neutral or acid pH, e.g. pH 3 to 7, especially pH 4 to 6.5, particularly pH 5 to 6.

The peroxide bleaching agent-containing compartment in the compositions of the invention moreover preferably contains a chelating agent capable of sequestering multivalent metal ions, e.g. EDTA or DTPA, preferably present as the sodium salt. Typically such chelating agents may be present at 0.01 to 5% wt., e.g. 0.5 to 2% wt.

Further components which may be included in the compositions according to the invention to enhance the stability thereof include water soluble polymers and low molecular weight organic compounds and surfactants, e.g. acrylate polymers, α, ω-unsaturated dicarboxylic acids, saturated dicarboxylic acids, polycarboxylic acids, phosphonic acids, tin salts, salicylic acid, hydroxyacids, glycerol, polyethylene glycols, monoglycerides, ionic and nonionic surfactants, etc. Such components will typically each be present at no more than about 30% wt., e.g. 1 to 5% wt.

The inclusion of a surfactant is especially preferred as the compositions can then readily be rinsed off the skin making it unnecessary to scrub or rub with a towel.

If desired, the compositions according to the invention may incorporate vitamins and/or antioxidants in order to reduce the risk of free radical damage to the skin. Typically such components include vitamins A, C and E, 2,6-di-tert.butyl-4-methoxylphenol (Butylhydroxyanisole or BHA), 2,6-di-tert.butyl-methyl-phenol (Butylhydroxytoluene or BHT) and glutathione. Again concentrations will typically be less than 10% wt., e.g. 0.01 to 5% wt.

Where the peroxide activator is an alkali, this can be any material capable of producing a pH of above 7 in an aqueous, peroxide-containing phase, preferably a pH of up to 10. Examples of suitable alkalis include sodium hydroxide, potassium hydroxide, tris(hydroxymethyl)aminomethane, sodium carbonate, sodium bicarbonate, ammonium carbonate, ammonium bicarbonate, ammonium hydroxide, alkylamines and aminoalcohols.

Where the activator is a di or multivalent cation it is preferably calcium, magnesium, strontium or zinc, preferably in a water soluble salt form, e.g. a carboxylic acid salt.

The individual components of the compositions should of course be acceptable for topical application to the skin, e.g. approved for cosmetic or pharmaceutical use.

As an alternative to physicochemical compartmentalization, compositions may be presented in a two-compartment dispenser (e.g. a spray bottle or a tube as is known for striped toothpaste) with a first peroxide bleaching agent composition in one compartment and the peroxide activator composition in the other compartment. Such two-compartment systems and dispensers containing these form further aspects of the invention.

Viewed from a further aspect the invention provides a kit comprising: a sealed container containing a skin bleaching composition according to the invention; and instructions for the use of said composition to bleach chemically tanned skin.

Viewed from a still further aspect the invention provides a method of cosmetic treatment to bleach chemically tanned skin, said method comprising applying to chemically tanned skin a composition according to the invention.

Viewed from a further aspect the invention provides a method of cosmetic treatment to bleach chemically tanned skin, said method comprising obtaining a kit according to the invention and applying said composition to chemically tanned skin.

The compositions of the invention are preferably in a ready-to-use form, i.e. so they can be applied directly to the skin without requiring further mixing following removal from the containers in which they are packaged.

The compositions of the invention are preferably emulsions, especially oil-in-water emulsions. The oil phase may be any topically tolerable oil or oil mixture but preferably comprises both hydrophobic and amphiphilic constituents. The use of liquid paraffin as at least one component of the oil phase is especially preferred.

The oil phase to aqueous phase ratio is preferably such that the emulsion composition has a creamy consistency rather than a paste or liquid like consistency.

The compositions desirably contain a surfactant to facilitate rinsing off of the composition after the treatment period, which typically will be 5 to 60 minutes, especially 15 to 30 minutes.

The compositions of the invention may be made by emulsifying a mixture of the individual ingredients. More preferably however they are produced by mixing hydrogen peroxide (in aqueous solution) with a pre-prepared emulsion, e.g. a skin cream, a body lotion, or a cream or unguent base for topical pharmaceutical application. Examples of suitable such preformed creams etc include Natusan pH 5.5 Cleansing Milk (Johnson & Johnson), Spenol (Nycomed Pharma AS, Oslo), Synergie Pure Deep Pore Wash (Laboratoire Garnier), Nivea Body Caring Milk (Beiersdorf), Unguentum M (Hermal, Reinbeck, Germany), ApoBase Creme (Alpharma AS, Oslo), Essex Cream (Schering-Plough, Belgium), and Locobase Cream (Yamanouchi Europe BV, Leiderdorp, Netherlands).

Typical ingredients of such creams, etc include water, liquid paraffin, alcohols, fatty acids, ester (e.g. glycerides), polymers (e.g. acrylates), glycols, gums, preservatives, surfactants, viscosity modifiers, buffers, fragrances, etc.

The peroxide bleaching agent is preferably added to a final content of 2 to 6% wt, more especially 3 to 5% wt, particularly 3.5 to 4.5% wt.

The compositions of the invention are surprisingly storage stable in terms of the maintenance of the peroxide bleaching agent content.

The invention will be described further with reference to the following non-limiting Examples.

### EXAMPLE 1

An aqueous solution of hydrogen peroxide was mixed with Natusan pH 5.5 Cleansing Milk from Johnson & Johnson using a pestle and mortar.

The composition of the product was:

Aqua, cetyl ethylhexanoate, isostearyl palmitate, pentaerythrityl tetraethylhexanoate, isononyl isononanoate, cyclomethicone, hexylene glycol, sorbitan stearate, peg-6 caprylic/capric glycerides, methylparaben, acrylates/c 10-30 alkyl acrylate crosspolymer, tromethamine, propylparaben, disodium EDTA, sucrose cocoate, hydrogen peroxide 4%.

### EXAMPLE 2

An aqueous solution of hydrogen peroxide was mixed with Spenol from Nycomed Pharma AS, Oslo, Norway, using a pestle and mortar. The composition of the product was:

Aqua, paraffinum liquidium, stearic acid, palmitic acid, propylene glycol, triethanolamine, cetyl alcohol, glyceryl stearate se, cera alba, methylparaben, propylparaben, hydrogen peroxide 4%.

### EXAMPLE 3

An aqueous solution of hydrogen peroxide was mixed with Synergie Pure Deep Pore Wash from Laboratoire Garnier, Paris, France, using a pestle and mortar. The composition of the product was

Aqua, alcohol denat, glycerine, sodium laureth sulphate, butylene glycol, sodium lauroyl sarcosinate, sodium diatomeae, triethanolamine, acrylates/c 10-30 alkyl acrylate crosspolymer, zinc pca, spiraea ulmaria, triclosan, salicylic acid, niacinamide, benzophenone-4, camphor, Cl42090, Peg-60, hydrogenated castor oil, polyethylene, tetrasodium EDTA, xanthan gum, imidazolidinyl urea, methylparaben, parfum, hydrogen peroxide 4%.

### EXAMPLE 4

An aqueous solution of hydrogen peroxide was mixed with Nivea body caring milk from Beiersdorf AG, Hamburg, Germany, using a pestle and mortar. The composition of the product was:

Aqua, paraffinum liquidium, glycerin cyclomethicone, isohexadecone, isopropyl palmitate, cera microcristallina, Peg-40, sorbitan perisostearate, polyglyceryl-3 diisostearate, sodium lactate, lactic acid, magnesium sulfate, prunus dulcis, tocopheryl acetate, lanolin alcohol, sodium bicarbonate, citric acid, lodopropynyl butylcarbamate, parfum, hydrogen peroxide 4%.

### EXAMPLE 5

An aqueous solution of hydrogen peroxide was mixed with Unguentum M from Hermal, Reinbeck, Germany, using a pestle and mortar. The composition of the product was:

Aqua, petrolatum, cetearyl alcohol, polysorbate 40, propylene glycol, glyceryl stearate, paraffinum liquidium, caprylic triglyceride, sorbic acid, silica, hydrogen peroxide 4%.

### EXAMPLE 6

An aqueous solution of hydrogen peroxide was mixed with ApoBase Creme from Alpharma AS, Oslo, Norway, using a pestle and mortar. The composition of the product was:

Aqua, petrolatum, paraffinum liquidium, cetearyl alcohol, Ceteth 20, phenoxyethanol, methyl-, ethyl-, propyl-, butyl-, and isobutylparaben, hydrogen peroxide 4%.

### EXAMPLE 7

An aqueous solution of hydrogen peroxide was mixed with Essex Cream from Schering-Plough, Belgium, using a pestle and mortar. The composition of the product was:

Aqua, petrolatum, cetearyl alcohol, paraffinum liquidium, Ceteth-20, sodium phosphate, p-chloro-m-cresol, phosphoric acid, hydrogen peroxide 4%.

### EXAMPLE 8

An aqueous solution of hydrogen peroxide was mixed with Locobase® cream from Yamanouchi Europe B.V., Leiderdorp, Netherlands, using a pestle and mortar. The composition of the product was:

Aqua, petrolatum, cetearyl alcohol, paraffinum liquidium, Ceteth-20, methylparaben, citric acid, sodium citrate, hydrogen peroxide 4%.

### EXAMPLE 9

Testing of stability:

The formulations of Example 5 and Example 7 were evaluated with regard to stability of hydrogen peroxide. The content of hydrogen peroxide was determined by direct titration with potassium permanganate. Both formulations did not show any sign of unstability after 6 weeks storage (the determined amount of hydrogen peroxide did not change by more than 2% from the initial value).

### EXAMPLE 10

### Clinical Trial

The formulations of Examples 2, 3, 4 and 5 were tested with regard to efficacy. A cream containing 16% DHA was used 3 times (over 3 days). The products from Examples 2, 3, 4 and 5 were then applied on the fourth day. The efficacy was scored on a scale 0-10 (10 best).

| Product | Efficacy score |
|---|---|
| Example 2 | 8 |
| Example 3 | 7 |
| Example 4 | 7-9.5 |
| Example 5 | 8.5-10 |

### EXAMPLE 11

### Sealed Container

The emulsion of Example 5 is filled into a blank opaque plastic “toothpaste” tube which is then heat sealed at the base (the end through which filling is effected).

The emulsions of Examples 1 to 4 and 6 to 8 may be similarly sealed within tubes.

The sealed tubes are then packed into card cartons together with instructions for the use of the emulsion to bleach chemically tanned skin, e.g. printed on the carton, on a label, on the tube, or on a package insert.

### EXAMPLE 12

Urea hydrogen peroxide addition compound (percarbamide) (3 g) from Aldrich is mixed into Unguentum Merck (97 g) using a pestle and mortar. The cream contains 3% hydrogen peroxide urea adduct. The product is filled in vials (20 g), sealed and kept in a refrigerator.

### EXAMPLE 13

Ammonium hydrogencarbonate (1.0 g) is suspended in methanol (50 ml). Phospholipon 100 H (soya phosphatidylcholine) from Rhône-Poulenc Rorer (100 mg) is added and the mixture is rapidly heated to dissolve the phospholipon and then stirred for 30 minutes at ambient temperature. The methanol is then evaporated using a rotary evaporator. The product is ammonium hydrogencarbonate coated with phospholipon 100 H.

Coated ammonium hydrogencarbonate (100 mg) is carefully mixed with Unguentum Merck containing 3% hydrogen peroxide (50 g). The product is filled into vials (10 g), sealed and kept in a refrigerator.

### EXAMPLE 14

Magnesium chloride (100 mg) is dissolved in water (5 ml). The solution is filled into a vial (Vial A). Hydrogen peroxide (3 g) is mixed into Unguentum Merck (92 g) using a pestle and mortar (Vial B).

The content in Vial A is added to Vial B and mixed with a wooden spatula before use.

### EXAMPLE 15

Liquid soap (1.0 g) (Zalo from Lilleborg, Norway) was mixed into Unguentum Merck (9.0 g) using a pestle and mortar.

This mixture could be removed from the skin using running water. Unguentum Merck without soap could not be completely removed this way.

### EXAMPLE 16

### Clinical Trial

A 42 year old woman had applied Piz Buin™ self-tanning cream to her whole body four times. A 4% wt hydrogen peroxide emulsion (e.g. as in Example 1) was applied giving extremely good results in terms of removal of the artificial tan. Up to 98% of tan was removed from arms, legs, stomach, chest and back using a single application of the emulsion. On the elbows, feet and hands the emulsion was applied twice. The result was absolutely satisfactory and no after effects were encountered. There was no hint of discomfort. The emulsion was left in place for 30 minutes. The same woman also tried other makes of self-tanning cream, e.g. L'Oreal, Matas, Delial and Superbraun and one which has a DHA content of 16% wt which is marketed as a pharmaceutical. Application of the emulsion of the invention was likewise effective at removing the chemically induced tan, again with no discomfort or after effects. Using the 16% DHA cream, applied twice, gave a much deeper tan; in this event two applications of the emulsion of the inventions were effective to remove the tan. The woman considered the emulsion of the invention to be the best tan-remover she had used.

### EXAMPLE 17

### Clinical Trial

A 50 year old woman applied the same self-tanning cream as was used by the 42 year old woman of Example 16, applying the cream in the same manner. A 4% wt hydrogen peroxide emulsion (e.g. as in Example 1) was applied giving very good results in terms of removal of the artificial tan. Up to 98% of tan was removed from the arms, legs, stomach, chest and back using a single application of the emulsion. On the elbows, feet and hands, the emulsion was applied twice. The woman indicated that about 99% of these areas were freed from tan after two applications of the emulsion. The emulsion was left in place for 30 minutes. There was no hint of discomfort or after effects. Once again the woman considered the emulsion of the invention to be the best tan-remover she had used.

### EXAMPLE 18

### Clinical Trial

A 16 year old woman applied self-tanning cream as described above for the 42 year old woman of Example 16. A 4% wt hydrogen peroxide emulsion (e.g. as in Example 1) was applied resulting in almost 100% removal of the tan from areas other than hard or dry skin. In areas of hard or dry skin, the woman used the emulsion of the invention twice resulting in almost total removal of the tan. The emulsion was left in place for 30 minutes and there was no hint of discomfort or after effects.

### EXAMPLE 19

### Clinical Trial

A 17 year old woman applied self-tanning cream and the emulsion of the invention as in the previous example. The result was the removal of about 98% of tan after a single application of the emulsion except in areas of hard or dry skin where a second application of the emulsion resulted in the tan being almost completely removed.

Similar results were also obtained for a 23 year old woman, a 30 year old man and a 50 year old man.

1. A kit comprising: a sealed container containing a ready-to-use skin bleaching composition comprising an emulsion containing 2 to 6% wt of a peroxide bleaching agent selected from the group consisting of hydrogen peroxide, organic peracids and salts thereof, hydrogen peroxide adducts, and metal peroxides, percarbonates and perboarates; and instructions for the use of said composition to bleach chemically tanned skin.

2. A kit as claimed in claim 1 wherein said peroxide bleaching agent is selected from hydrogen peroxide and hydrogen peroxide adducts.

3. A kit as claimed in claim 1 wherein said peroxide bleaching agent is hydrogen peroxide.

4. A kit as claimed in claim 1 wherein the pH of said emulsion is from 4.5 to 6.5.

5. A kit as claimed in claim 1 wherein said container is a plastic tube or bottle.

6. A method of cosmetic treatment to bleach chemically tanned skin, said method comprising obtaining a kit according to claim 1 and applying said composition to chemically tanned skin.

7. A method of cosmetic treatment to bleach chemically tanned skin, said method comprising applying to chemically tanned skin a topically acceptable emulsion which comprises 2 to 6% wt of a peroxide bleaching agent selected from the group consisting of hydrogen peroxide, organic peracids and salts thereof, hydrogen peroxide adducts, and metal peroxides, percarbonates and perboarates, peroxide and has a neutral or acid pH.

8. A method of cosmetic treatment to bleach chemically tanned skin, said method comprising applying to chemically tanned skin a topically acceptable emulsion which is essentially free of alkali hydrogen peroxide activators and comprises 2 to 6% wt a peroxide bleaching agent selected from the group consisting of hydrogen peroxide, organic peracids and salts thereof, hydrogen peroxide adducts, and metal peroxides, percarbonates and perborates peroxide.

9. A kit comprising: a sealed container containing a composition for the bleaching of chemically tanned skin containing a peroxide bleaching agent and a physiologically tolerable carrier, characterized in that physiologically tolerable carrier, characterized in that said peroxide bleaching agent is selected from organic peracids and salts thereof, hydrogen peroxide adducts, and metal peroxides, percarbonates and perborates; and instructions for use in treatment of the skin to bleach chemically tanned skin.

10. A kit as claimed in claim 9 wherein said peroxide bleaching agent is a hydrogen peroxide-urea adduct.

11. A kit as claimed in claim 9 wherein said peroxide bleaching agent is a group I or II peroxide, percarbonate or perborate.

12. A kit as claimed in claim 9 wherein said composition is in emulsion form.

13. A kit as claimed in claim 9 wherein said composition contains 2 to 6% wt of said peroxide bleaching agent.

14. A kit as claimed in claim 9 wherein said composition has a pH of 4.5 to 6.5.

15. A kit as claimed in claim 9 wherein said composition is packaged in a sealed plastic tube or bottle.

16. A method of cosmetic treatment to bleach chemically tanned skin, said method comprising obtaining a kit according to claim 9 and applying said composition to chemically tanned skin.

17. A ready-to-use skin bleaching composition for bleaching chemically tanned skin, said composition being compartmentalized having a continuous compartment and a discontinuous compartment with one of said compartments containing a peroxide bleaching agent and the other of said compartments containing a peroxide activator.
